(19) United States
(12) Patent Application Publication
(10) Pub. No.: US 2026/0364237 A1
(43) Pub. Date: Nov. 19, 2026

(54) BLOOD TREATMENT APPARATUS WITH COMPENSATION OF CHANGES IN THE AMBIENT TEMPERATURE

(71) Applicant: Fresenius Medical Care Deutschland GmbH, Bad Homburg (DE)

(72) Inventor: Olaf Nicholas, Bad Homburg (DE)

(21) Appl. No.: 19/158,737

(22) PCT Filed: Feb. 21, 2024
(86) PCT No.: PCT/EP2024/054425

§ 371 Date: Aug. 21, 2025

(30) Foreign Application Priority Data
Feb. 23, 2023 (DE) 102023104402.8

(51) Int. Cl. A61M 1/16

(52) U.S. Cl. CPC A61M 1/1664; A61M 2205/3372; A61M 2205/50

(57) ABSTRACT

A control device or closed-loop control device is configured to control or control in a closed-loop manner the operation of a blood treatment apparatus for treating a patient during a blood treatment session, in which the blood treatment apparatus is connected to an extracorporeal blood circuit and a blood treatment device. The blood treatment apparatus comprises a dialysis liquid inlet line and a dialysate outlet line, a heating device for heating the dialysis liquid, at least a first temperature sensor arranged downstream of the blood treatment device and/or downstream of the dialysis liquid inlet line, for determining a first temperature value (T₁) of a first liquid previously advanced along the dialysis liquid inlet line. The control device or closed-loop control device is configured to increase or set the temperature of the dialysis liquid using the heating device based on the first temperature value determined by the first temperature sensor.

### CROSS-REFERENCE TO RELATED APPLICATIONS

The present application is the national stage entry of International Patent Application No. PCT/EP2024/054425, filed Feb. 21, 2024, and claims priority to Application No. DE102023104402.8, filed in the Federal Republic of Germany on Feb. 23, 2023, the contents of which are hereby incorporated by reference.

### TECHNICAL FIELD

The present disclosure relates to a control device or closed-loop control device, to a blood treatment apparatus, to a digital storage medium, to a computer program product, and to a computer program.

### BACKGROUND

Known from practice are blood treatment apparatuses which apply a treatment liquid to the patient by a pump, often, as in the example of the dialysis apparatus, via the extracorporeal blood circuit. In blood treatment apparatuses, such as dialysis apparatuses, treatment liquid is additionally or alternatively supplied to, e.g., a blood filter through which blood flows. Since the temperature of the treatment liquid conveyed in both of the aforementioned cases may have an influence on the temperature of the blood to be reinfused to the patient, a heating device may be provided for heating or tempering the treatment liquid.

### SUMMARY

It is an aspect of the present disclosure to specify a further control device or closed-loop control device and a further blood treatment apparatus. A digital storage medium, a computer program product, and a computer program are also to be specified.

The present disclosure relates to a control device or closed-loop control device configured to control or control in a closed-loop manner the operation of a blood treatment apparatus for treating a patient during a blood treatment session, in which the blood treatment apparatus is connected to an extracorporeal blood circuit and a blood treatment device, e.g., a dialyzer or a blood filter.

The blood treatment apparatus further comprises a dialysis liquid inlet line arranged to supply dialysis liquid to the blood treatment device during the blood treatment session, and a dialysate outlet line arranged to discharge dialysate, (e.g., spent dialysis liquid), from the blood treatment device during the blood treatment session.

A heating device for heating the dialysis liquid before or during the blood treatment session is also comprised by the blood treatment apparatus.

The blood treatment apparatus further comprises one or several temperature sensors. These may be temperature gauges or devices by which temperature can be estimated.

At least a first of these temperature sensors, which is arranged downstream of the blood treatment device and/or downstream of the dialysis liquid inlet line, serves to determine a first temperature value of a first liquid previously advanced along the dialysis liquid inlet line towards the blood treatment device.

In this, the control device or closed-loop control device is configured to increase or set the temperature of the dialysis liquid by the heating device based on the first temperature value determined by the first temperature sensor.

A blood treatment apparatus which is prepared for use with an extracorporeal blood circuit and a blood treatment device, e.g., a dialyzer or a blood filter, for the purpose of treating blood of a patient during a blood treatment session is proposed by the present disclosure.

The blood treatment apparatus according to the present disclosure comprises at least one receptacle for releasably receiving at least one section of the extracorporeal blood circuit on the blood treatment apparatus.

The blood treatment apparatus according to the present disclosure further comprises at least one receptacle for releasably receiving at least one section of the blood treatment device on the blood treatment apparatus.

The blood treatment apparatus according to the present disclosure further comprises a dialysis liquid inlet line arranged to supply dialysis liquid to the blood treatment device during the blood treatment session, and a dialysate outlet line arranged to discharge dialysate, (e.g., spent dialysis liquid), from the blood treatment device during the blood treatment session.

The blood treatment apparatus further comprises one or several temperature sensors, which may be temperature gauges or devices for estimating a temperature. At least a first temperature sensor of these temperature sensors is arranged downstream of the blood treatment device and/or downstream of a dialysis liquid inlet line. The first temperature sensor serves to determine a first temperature value of a first liquid flowing through, or resting in, the dialysis liquid inlet line.

A heating device serves for heating the dialysis liquid before or during the blood treatment session and is also comprised by the blood treatment apparatus according to the present disclosure.

The blood treatment apparatus according to the present disclosure further comprises a control device or closed-loop control device according to the present disclosure, by which the operation of the blood treatment apparatus may be controlled or controlled in closed-loop manner.

A digital, e.g., non-volatile storage medium, according to the present disclosure, e.g., in the form of a machine-readable carrier, e.g., in the form of a diskette, memory card, CD, DVD, EPROM, FRAM (Ferroelectric RAM) or SSD (Solid-State-Drive), e.g., with electronically or optically readable control signals, may interact with a programmable computer system such that a conventional control device or closed-loop control device is reprogrammed into a control device or closed-loop control device according to the present disclosure. Alternatively or additionally, a conventional blood treatment apparatus is reprogrammed into a blood treatment apparatus according to the present disclosure.

A computer program product according to the present disclosure comprises a volatile or transient program code or one stored on a machine-readable carrier or a signal wave, with which a conventional control device or closed-loop control device is reprogrammed into a control device or closed-loop control device according to the present disclosure when the computer program product runs on a computer. Alternatively or additionally, a conventional blood treatment apparatus is reprogrammed into a blood treatment apparatus according to the present disclosure.

A computer program product may be understood according to the present disclosure, for example, as a computer program stored on a carrier, an embedded system being a comprehensive system with a computer program (e.g., an electronic device with a computer program), a network of computer implemented computer programs (e.g., client/server-system, a cloud computing system etc.), or a computer on which a computer program is loaded, runs, is stored, is being executed or developed.

The term “machine-readable carrier” as used herein, refers in certain embodiments of the present disclosure to a carrier, which contains data or information interpretable by software and/or hardware. The carrier may be a data carrier, such as a diskette, a CD, DVD, a USB stick, a flashcard, an SD card or the like, as well as any other storage referred to herein or any other storage medium referred to herein.

A computer program according to the present disclosure comprises a program code with which a conventional control device or closed-loop control device is reprogrammed into a control device or closed-loop control device according to the present disclosure, when the computer program is running on a computer. Alternatively or additionally, a conventional blood treatment apparatus is reprogrammed into a blood treatment apparatus according to the present disclosure.

Embodiments according to the present disclosure may comprise one, several or all of the features mentioned below in any combination unless the person skilled in the art considers the particular combination to be technically impossible.

In all of the aforementioned and following statements, the use of the expression “may be” or “may have” and so on, is to be understood synonymously with “preferably is” or “preferably has”, and so on respectively, and is intended to illustrate embodiments according to the present disclosure.

Whenever numerical words are mentioned herein, the person skilled in the art shall recognize or understand them as indications of numerical lower limits. Hence, unless this leads to a contradiction evident for the person skilled in the art, the person skilled in the art shall comprehend for example “one” (or “a/an”) as encompassing “at least one”. This understanding is also equally encompassed by the present disclosure as the interpretation that a numerical word, for example, “one” (or “a/an”) may alternatively mean “exactly one”, wherever this is evidently technically possible in the view of the person skilled in the art. Both of these understandings are encompassed by the present disclosure and apply to all numerical words used herein.

The person skilled in the art shall understand spatial information like, e.g., “top”, “bottom”, “left” or “right”, whenever they are herein mentioned, as a spatial indication with reference to the alignment in the figures appended hereto and/or during use. “Bottom” is closer to the geocenter or to the lower edge of the figure than “top”.

When “determining” is mentioned herein, e.g., that of data and/or temperature values, this may be or encompass examining an existence or non-existence, a measuring, setting, detecting, recording, collecting, evaluating, processing, comparing, estimating, rating or appraising, deducing, calculating, obtaining, reaching or achieving, and/or recognizing.

Whenever an embodiment is mentioned herein, it represents an exemplary embodiment according to the present disclosure which is not to be understood as limiting.

When it is disclosed herein that the subject-matter according to the present disclosure comprises one or several features in a certain embodiment, it is also respectively disclosed herein that the subject-matter according to the present disclosure does, in other embodiments, likewise according to the present disclosure, explicitly not comprise this or these features, for example, in the sense of a disclaimer.

Therefore, for every embodiment mentioned herein it applies that the converse embodiment, e.g., formulated as negation, is also disclosed.

When programmed or configured is mentioned herein, then these terms may in some embodiments be interchangeable.

When a signal communication or communication connection between two components is mentioned herein, this may be understood to mean a connection that exits during use. It may also be understood that a preparation for such a (wired, wireless or otherwise implemented) signal communication exists, for example by coupling both components, for instance by pairing, etc.

Pairing is a process that takes place in connection with computer networks in order to establish an initial link between computer units for the purpose of communication. The best-known example of this is the establishing of a Bluetooth connection, by which various devices (e.g., smartphone, headphones) are connected to each other. Pairing is sometimes also referred to as bonding.

The control device or closed-loop control device may prompt all or substantially all of the method steps to be executed. The method according to the present disclosure may be executed substantially or completely by the control device. The method may be partially executed by the control device, e.g., those features which do not require or involve human intervention and/or a provision may be executed by the control device. The control device may be used purely as a control device or also as a closed-loop control device.

In several embodiments, the control device or closed-loop control device is present in or on the blood treatment apparatus, for instance together with other components or devices of the blood treatment apparatus in a common housing of the blood treatment apparatus.

In some embodiments, the control device or closed-loop control device according to the present disclosure is configured to increase or set the temperature by the heating device additionally based on a second temperature value. The second temperature value may be, or have been, determined by a second temperature sensor of the plurality of temperature sensors on the first liquid upstream of the blood treatment device and/or upstream of the dialysate outlet line.

In several embodiments, the first and/or the second temperature value is or was determined prior to the start of the blood treatment session.

The first and/or the second temperature value may be stored in the control device or closed-loop control device or in a storage device suitable or provided for this purpose.

In some embodiments, the first and/or the second temperature value is or was determined during the blood treatment session.

In several embodiments, the first or the second temperature value, respectively, is or was determined while the dialysis liquid inlet line and the dialysate outlet line are or were connected in fluid communication with each other, for example by a short circuit line, excluding or bypassing the blood treatment device.

In some embodiments, the first and/or the second temperature value is or was determined while the dialysis liquid inlet line and the dialysate outlet line are or were not arranged excluding of or bypassing the blood treatment device, but are respectively connected to the blood treatment apparatus, for example in fluid communication.

In several embodiments of the control device or closed-loop control device according to the present disclosure, when, or for, determining the first and/or the second temperature value no two liquids, being different from each other, flow through the blood treatment device, or are present therein.

In some embodiments, no blood as a second liquid flows or is present therein.

In some embodiments of the control device or closed-loop control device, a deviation of the first temperature value of the first liquid from a setpoint value for that liquid at this point is determined.

In several embodiments of the control device or closed-loop control device, it is configured to calculate a calculated temperature value based on both the first temperature value and the second temperature value of the first liquid, in order to determine a deviation of the calculated temperature value on the blood treatment device from a setpoint value for the temperature at this point and to prompt the increase or setting of the dialysis liquid temperature by the heating device based on the deviation of the calculated temperature value at the blood treatment device from a setpoint value for the blood treatment device.

In some embodiments, a setpoint temperature for the temperature value at the dialyzer, which is required for determining a deviation of the temperature value at the dialyzer from the same, and/or a setpoint temperature for the temperature value at the first temperature sensor, which is required for determining a deviation of the measured first temperature value from the setpoint temperature at the first temperature sensor, are entered by the user, for example via an input interface of the blood treatment apparatus. Alternatively, these setpoint values are stored in a storage device provided for this purpose and may be retrieved from there by the control device or closed-loop control device.

In some embodiments, the calculated temperature value at the blood treatment device is or was determined or calculated based on the first and/or second temperature value as well as on a temperature coefficient, determined for the dialysis liquid inlet line or for a first line segment, e.g., extending outside the blood treatment apparatus, e.g., for its length, and/or for the dialysate outlet line, or for a second line segment, e.g., extending outside the blood treatment apparatus, e.g., for its length.

The calculation of the calculated temperature value at the blood treatment device or dialyzer gives an estimation of the actual temperature of the first liquid as it flows through the blood treatment device. This may be used to determine a deviation between the estimated temperature at the blood treatment device and its setpoint value at this point. The deviation is used to prompt, according to the present disclosure, the increasing or the setting of the dialysis liquid temperature by the heating device based on the deviation of the calculated temperature value from its setpoint value.

In this, the following applies to a calculated temperature value T_{rech} in the dialyzer in an embodiment

T_{rech}=T2−L1*Tₖ

or

T_{rech}=T1+L2*Tₖ

and for the temperature at the first temperature sensor

T1=T2−((L1+L2)*Tₖ).

In several embodiments, the control device or closed-loop control device is configured to determine the temperature coefficient Tₖ.

If a linear temperature drop is assumed for the respective line length L1, L2 of the line segments, a temperature coefficient Tₖ in the unit [°C/m] can be defined for these, e.g., as follows:

$${T}_{k} = \frac{( T 2 - T 1 )}{L 1 + L 2} ,$$

wherein the following applies:
T1 measured first temperature value from the first temperature sensor downstream of the dialyzer
T2 measured second temperature value from the second temperature sensor upstream of the dialyzer
L1 length of the line segment, e.g., outside the blood treatment apparatus upstream of the dialyzer
L2 length of the line segment, e.g., outside the blood treatment apparatus downstream of the dialyzer

In some embodiments, the control device or closed-loop control device is configured to determine the temperature coefficient relative to one, two or more, e.g., predetermined, flow rates of the first liquid into or through the blood treatment device. Alternatively, the temperature coefficient is or was determined for one, two or more, e.g., predetermined, flow velocities of the first liquid, respectively, excluding or bypassing the blood treatment device within a short circuit, for example within a short circuit line, between the dialysis liquid inlet line and the dialysate outlet line.

If a linear relationship between temperature drop and flow is assumed, the following applies for a reference flow of 500 ml/min:

$${T}_{k flow - compensated} = {T}_{k} * ( \frac{500 ml / min}{actual flow} )$$

The influence of an ambient temperature value T_{Umg} is assumed to be homogeneous in these calculations in certain embodiments.

In some embodiments, the control device or closed-loop control device according to the present disclosure is configured to increase or decrease the temperature of the dialysis liquid, to which it is heated using the heating device during or before the blood treatment session, by the difference between the first temperature value or the calculated temperature value and, respectively, the setpoint for the temperature at those points.

In several embodiments of the control device or closed-loop control device, only the first temperature value downstream of the blood treatment device is determined.

In some embodiments, the control device or closed-loop control device is configured to further take into account a determined ambient temperature value when increasing or setting the temperature up to which the dialysis liquid is heated by the heating device during or before the blood treatment session. The ambient temperature value may be, or have been, determined for example by an ambient temperature sensor, e.g., a temperature sensor of the blood treatment apparatus.

In several embodiments, the blood treatment apparatus according to the present disclosure further comprises a second temperature sensor among the plurality of temperature sensors for determining a second temperature value of the first liquid upstream of the blood treatment device or upstream of the dialysate outlet line.

In some embodiments, the blood treatment apparatus further comprises, or is connected to, an ambient temperature sensor. The ambient temperature sensor is suitable and/or provided for determining an ambient temperature value of the blood treatment apparatus.

In certain embodiments, the environment temperature sensor is in signal communication with the control device or closed-loop control device and can transmit the measured ambient temperature value to it. Alternatively, the control device or closed-loop control device can be used to retrieve this temperature value from the ambient temperature sensor.

In several embodiments, the blood treatment apparatus is embodied as a dialysis apparatus, hemodialysis apparatus, hemofiltration apparatus or hemodiafiltration apparatus, e.g., as an apparatus for the acute kidney replacement therapy, the chronic kidney replacement therapy or for the continuous kidney replacement therapy (CKRT).

In some embodiments, the present disclosure is not limited to the use of a blood treatment apparatus which uses a dialyzer for blood treatment. Its use in other fields of medical technology, which does not serve the treatment of renally-impaired patients, is also encompassed by the present disclosure.

In some embodiments, further device-internal influences, e.g., temperature influences, are included in the calculation as described herein, while in others they are disregarded. Such consideration may be made, for example, in determining the temperature coefficient TR. Thus, a series of temperature coefficients Tₖ may be determined at different, predetermined environment conditions and above all environment temperatures, respectively, and optionally stored in, e.g., the storage device.

In several embodiments, values, setpoints, coefficients, and the like disclosed herein may optionally be determined or calculated by the control device or closed-loop control device within the scope of the present disclosure, such as by a corresponding calculating device comprised by, or connected to, the control device or closed-loop control device. They may in each case alternatively be taken from a storage device by the latter. There, these values, setpoints, coefficients and the like may have been stored after they were first determined by the control device or closed-loop control device or the computing device in a previous step or at an earlier point in time.

When method steps are mentioned herein, it is provided in several embodiments that they occur before or after a treatment of the patient, such as while the patient is not connected to the apparatus, e.g., via an extracorporeal blood circuit or the like, or that a treatment has not yet begun or has already ended.

In some embodiments, no body temperature of the patient and/or temperature correlating therewith is measured or determined and/or the temperature sensors are not arranged for this purpose. In several embodiments, the body temperature is not included in the calculation or determination performed or initiated by the control device or closed-loop control device or other component, nor in the controls.

In several embodiments, the control device or closed-loop device does not take into account the patient's body temperature and/or a temperature correlated therewith when or for adjusting or increasing the temperature of the dialysis liquid using the heating device.

In some embodiments, no variable, which is a variable characteristic of the heat transfer through the dialyzer, is taken into account in the calculation or determination performed or initiated by the control device or closed-loop control device or by another component, nor in the controls.

Some or all embodiments according to the present disclosure may comprise one, several or all of the advantages mentioned supra and/or in the following.

One advantage of the present disclosure may be that the heating power of the heating device is subject to temperature-based control, thus it may be ensured that the temperature at the predetermined points does not exceed a maximum temperature and/or does not fall below a setpoint temperature. Corresponding adjustments of conveyance rates of the pump for conveying the treatment liquid with regard to the aforementioned temperature values may be omitted and the patient may be protected from the consequences of using a treatment liquid having an unsuitable temperature.

The temperature at the temperature sensors or a heat loss of the treatment liquid between the first and the second temperature sensor may according to the present disclosure be determined by simple means and is, hence, known, thus temperature differences may be advantageously detected and the heating device may be controlled in a closed-loop manner accordingly.

Another advantage of the present disclosure may be that existing systems may be easily retrofitted by a software update, since already existing sensors of the blood treatment apparatus as well as its control device or closed-loop control device may be used.

### BRIEF DESCRIPTION OF THE FIGURES

In the following, the present disclosure will be merely exemplarily described with reference to the accompanying figures. In them, same reference numerals denote identical or similar components. In the figures, the following applies:

FIG. 1 shows schematically simplified a fluid line diagram of a blood treatment apparatus in a first embodiment;

FIG. 2 shows a section of the fluid line diagram of FIG. 1, in which instead of the blood treatment device a short circuit line connects dialysis liquid inlet line and dialysate outlet line to each other; and

FIG. 3 shows schematically simplified a control device or closed-loop control device with temperature data streams.

### DETAILED DESCRIPTION

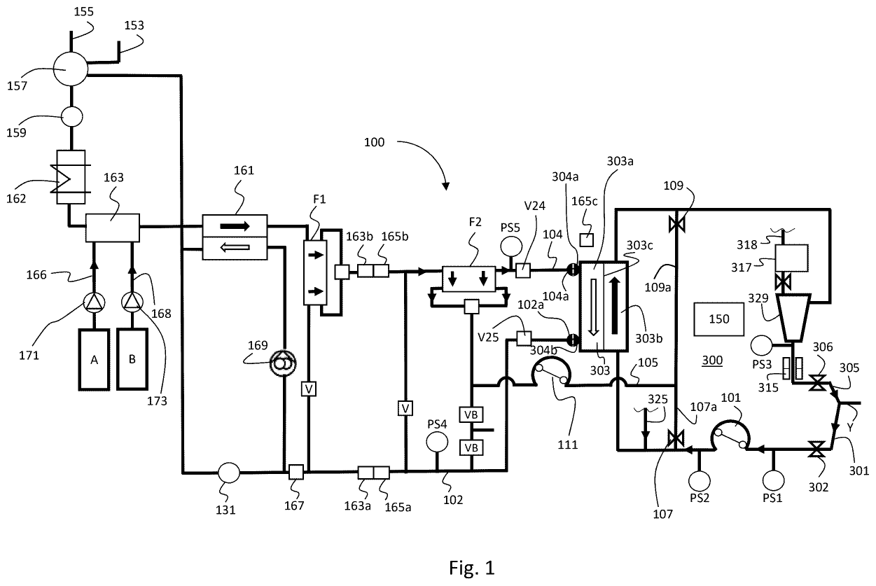

FIG. 1 shows a fluid line diagram of a blood treatment apparatus 100 according to the present disclosure in a first embodiment. In FIG. 1, the blood treatment apparatus is represented only through single components being partially highly schematically simplified.

The blood treatment apparatus 100, which is shown in an at least partially equipped condition of use, is connected to an extracorporeal blood circuit 300, which can be connected to the vascular system of the patient, not shown, for treatment by double-needle access, or when using, e.g., an additional Y-connector (reference numeral Y), as shown in FIG. 1, by single-needle access wherein said extracorporeal blood circuit 300 is optionally not part of the blood treatment apparatus 100; however it is part thereof in other embodiments. The blood circuit 300 may be present, optionally in sections thereof, in or on a blood cassette.

Pumps, actuators and/or valves in the area of the blood circuit 300 are connected in signal communication to the blood treatment apparatus 100 or to a control device or closed-loop control device 150, respectively, in the event that controlling or controlling in a closed-loop manner is required.

The blood circuit 300 comprises (or is connected to) an arterial patient tube clamp 302 and an arterial connection needle (not shown in FIG. 1) of an arterial section or of an arterial patient line, blood withdrawal line or first line 301.

The blood circuit 300 also comprises (or is connected to) a venous patient tube clamp 306 and a venous connection needle (not shown in FIG. 1) of a venous section, a venous patient line, a blood return line or a second line 305.

A blood pump 101 is provided in or at the first line 301, an optional substitute fluid pump 111 is connected to, e.g., a dialysis liquid inlet line 104 for conveying fresh dialysis liquid, which is filtered (substitute fluid) in a filtering step (filter F2).

An optional substitute fluid line 105 may be fluidically connected, for example, to a dialysis liquid inlet line 104. Using the substitute fluid pump 111, substitute fluid may be introduced by pre-dilution, via an optional pre-dilution valve 107, or by post-dilution, via a an optional post-dilution valve 109, via optional, associated lines 107a or 109a into line sections, for example into the arterial line section 301 or into the venous line section 305 (here between a blood chamber 303b of a blood filter 303 and a venous air separation chamber or venous blood trap 329) of the blood circuit 300.

The blood filter 303 comprises the blood chamber 303b connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102, which guides dialysate, (e.g., spent dialysis liquid), leading away from the dialysis liquid chamber 303a. Suitable connectors 104a, 102a on the dialysis liquid inlet line 104 or on the dialysate outlet line 102 on the one hand, and dialysate ports 304a, 304b of the blood filter 303 on the other hand, which may be connected, e.g., releasably, to each other, serve for this purpose.

Dialysis liquid chamber 303a and blood chamber 303b are separated from each other by a mostly semipermeable membrane 303c. It represents the partition between the blood side with the extracorporeal blood circuit 300 and the machine side with the dialysis liquid circuit or dialysate circuit, which is shown in FIG. 1 to the left of the membrane 303c.

The arrangement of FIG. 1 comprises an optional detector 315 for detecting air and/or blood. The arrangement of FIG. 1 optionally further comprises one or two pressure sensors PS1 (upstream of the blood pump 101) and PS2 (downstream of the blood pump 101, and the arrangement measures the pressure upstream of the blood filter 303 (“pre-hemofilter”)) at the points shown in FIG. 1. Further pressure sensors may be provided, such as pressure sensor PS3 downstream of venous bubble trap 329.

An optional single-needle chamber 317 is used in FIG. 1 as a buffer container and/or compensating reservoir in a single-needle procedure in which the patient is connected to the extracorporeal blood circuit 300 using only one of the two blood lines 301, 305.

An addition site 325 for Heparin or for another, e.g., local, anticoagulant may optionally be provided.

On the left in FIG. 1, an optional mixing device 163 is shown, which provides a predetermined mixture for the respective solution from the containers A (for A-concentrate via concentrate supply 166) and B (for B-concentrate via concentrate supply 168) for use by the blood treatment apparatus 100. The solution contains water from the water source 155 (on-line, e.g., as reverse osmosis water or from bags), which water is heated, e.g., in the heating device 162.

An optional pump 171, which can be referred to as concentrate pump or sodium pump, is fluidically connected to the mixing device 163 and to a source of sodium, such as the container A, and/or conveys out of the mixing device 163. An optional pump 173 associated with container B, such as for bicarbonate, can be seen.

Furthermore, FIG. 1 shows a drainage 153 for the effluent. An optional heat exchanger 157 and an optional first flow pump 159, which is suitable for degassing, complete the arrangement shown.

The optional pressure sensor PS4 may be provided downstream of the blood filter 303 on the water side, but, e.g., upstream of an optional ultrafiltration pump 131 in the dialysate outlet line 102 for measuring the filtrate pressure or membrane pressure of the blood filter 303.

The ultrafiltration pump 131 provides a means for accurately removing, from the balanced circuit, a volume of liquid being specified by the user and/or by the control device or closed-loop control device 150.

Blood leaving the blood filter 303 flows through an optional venous bubble trap 329, which may comprise a deaeration device 318 and may be in fluid communication with the pressure sensor PS3.

The exemplary arrangement shown in FIG. 1 comprises the control device or closed-loop control device 150. The latter may be in a wired or wireless signal connection with any of the components mentioned herein (e.g., with the blood pump 101) in order to control or control in a closed-loop manner the blood treatment apparatus 100.

By using the device for on-line mixing of the dialysis liquid, a variation of its sodium content, controlled by the control device or closed-loop control device 150, is possible within certain limits. For this purpose, e.g., the measured values determined by the conductivity sensors 163a, 163b may be taken into account. Should an adjustment of the sodium content of the dialysis liquid (sodium concentration) or of the substitute fluid turn out to be necessary or desired, this can be done by adjusting the conveying rate of the sodium pump 171.

In addition, the treatment apparatus 100 comprises devices for conveying fresh dialysis liquid and dialysate. An optional first valve V24 may be provided between the first flow pump 159 and the blood filter 303, which first valve V24 opens or closes the inflow towards the blood filter 303 on the inlet side. A second, optional flow pump 169 which conveys dialysate to the drainage 153 is provided, e.g., downstream of the blood filter 303. A second valve V25 may be provided between the blood filter 303 and the second flow pump 169, which second valve V25 opens or closes the outflow on the outlet side.

Furthermore, the blood treatment apparatus 100 optionally comprises a device 161 for balancing the flow flowing into and out of the dialyzer 303 on the machine side. The device 161 for balancing can be arranged in a line section between the first flow pump 159 and the second flow pump 169.

Sensors such as the optional conductivity sensors 163a, 163b serve to determine the conductivity, which in some embodiments is temperature-compensated, as well as the fluid flow upstream and downstream of the dialyzer 303.

Temperature sensors, including the first temperature sensor 165a and/or the second temperature sensor 165b may be provided singly or in groups. Temperature values supplied by them can be used during treatment of the patient for determining a temperature-compensated conductivity, during blood treatment for determining at least one temperature value of the dialysate (downstream of the dialyzer 303) and/or at least one temperature value of the dialysis liquid (upstream of the dialyzer 303), or, according to the present disclosure, for determining at least a first temperature value T₁ of the liquid flowing downstream of and out of the dialyzer (which liquid could be referred to as dialysate during the blood treatment, which is also done herein in accordance therewith) and/or at least a second temperature value T₂ of the liquid flowing upstream of and into the dialyzer (which liquid could be referred to as dialysis liquid during the blood treatment, which is also done herein in accordance therewith).

A leakage sensor 167 is optionally provided. The leakage sensor 167 may be provided also at a different position.

Further flow pumps may also be provided in addition or alternatively to, e.g., the one with the reference numeral 169.

A number of optional valves are each denoted with V in FIG. 1; by-pass valves with VB.

A pressure sensor P25 for measuring the pressure in the dialysis liquid inlet line 104 may be provided.

The control device or closed-loop control device 150 determines in several embodiments the electrolyte balance and/or liquid balance based on the measured values from the aforementioned optional sensors.

Filters F1 and F2 can be provided being connected in series.

Even when using non-pure water, the filter F1 exemplarily serves herein to generate sufficiently pure dialysis liquid by the mixing device 163, which then flows through the blood filter 303, e.g., using the countercurrent principle.

The filter F2 exemplarily serves herein to generate sterile or sufficiently filtered substitute fluid from the sufficiently pure dialysis liquid leaving the first filter F1, by filtering, e.g., pyrogenic substances. This substitute fluid may then be safely added to the extracorporeally flowing blood of the patient and thus ultimately to the patient's body.

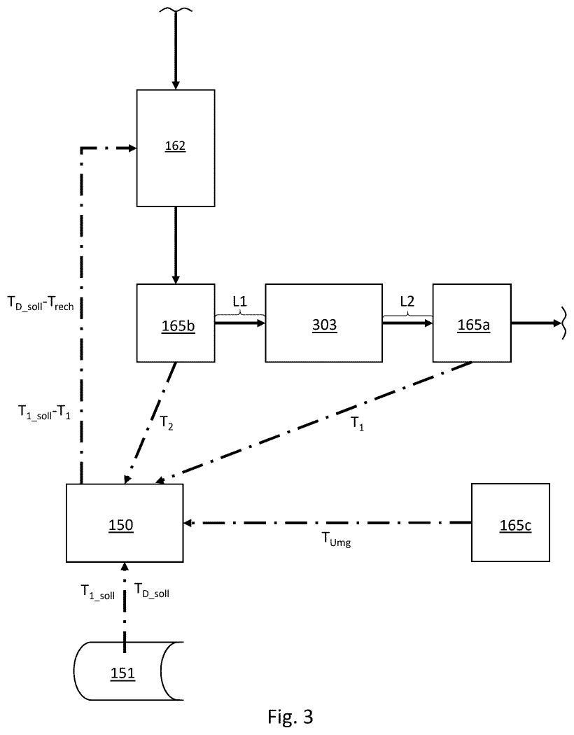

An optional ambient temperature sensor 165c, which may be part of the blood treatment apparatus 100, may be in signal communication with the control device or closed-loop control device 150 thereof in order to determine an ambient temperature value T_{Umg} (see FIG. 3). The control device or closed-loop control device 150 may be configured such that to further take into account this determined ambient temperature value T_{Umg} When increasing or setting the temperature.

The ambient temperature sensor 165c may also be provided separately from the blood treatment apparatus 100, but still in signal communication with the control device or closed-loop control device 150 thereof.

Although the blood treatment apparatus 100 is optionally shown in FIG. 1 as an apparatus for hemodiafiltration, nevertheless hemodialysis apparatuses are also covered by the present disclosure, even though not specifically represented in a figure.

The arrows shown in FIG. 1 generally indicate the flow direction in FIG. 1.

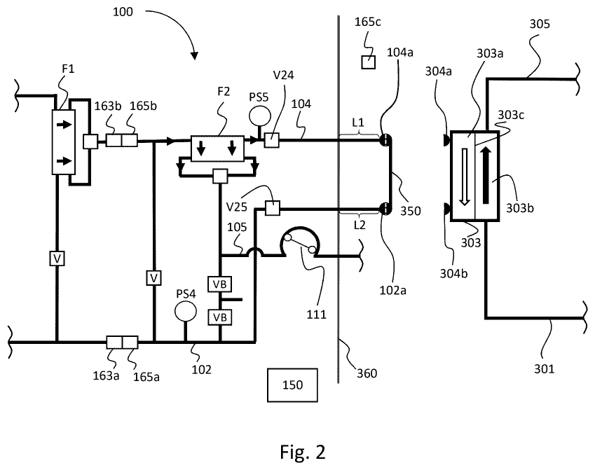

FIG. 2 shows a section of the fluid line diagram of FIG. 1, in which instead of the blood filter 303, a short circuit line 350 connects dialysis liquid inlet line 104 and dialysate outlet line 102, which is equivalent to bypassing the blood filter 303. Such a short circuit may be used for example beforehand an upcoming blood treatment in order to first check all relevant components of the blood treatment apparatus 100 regarding their functionality.

Reference is made to the description with regard to FIG. 1.

In the example of FIG. 2, the dialyzer or blood filter 303 is, together with the extracorporeal blood circuit 300 and its pumps, actuators and/or valves, present in a not yet equipped state and therefore shown separately from the hydraulics of the blood treatment apparatus 100.

A housing wall 360 of the blood treatment apparatus 100 is indicated by a double bar. According to the present disclosure, it is assumed that a temperature of the dialysis liquid or the dialysate inside the blood treatment apparatus 100 (e.g., to the left of the housing wall 360) does not change, while the dialysis liquid or dialysate is exposed to an ambient temperature along the tube lines or line segments, which run over determinable lengths L1, L2 outside the blood treatment apparatus 100 (to the right of the housing wall 360 in FIG. 2) over lengths L1, L2 that are determinable. The ambient temperature may effect a change in temperature of the dialysis liquid or of the dialysate. A change in temperature of the dialysis liquid (e.g., of the liquid flowing toward or into the dialyzer) within the treatment apparatus 100 does naturally occur under real conditions, but it is so minor it can be neglected with regard to the considerations of the present disclosure.

In some embodiments, the blood treatment apparatus 100 is calibrated. For this purpose, optionally a predetermined setpoint flow through the dialyzer 303, but in any case a predetermined setpoint temperature T_{D_soll} for the temperature of the dialysis liquid or the dialysate at the dialyzer 303, or when flowing through it, is to be specified. The latter may be stored in a storage device for the control device or closed-loop control device 150 or may be entered by the user on, e.g., the blood treatment apparatus 100.

After a start-up phase, there may be determined, e.g., measured, for example a first temperature T₁ (here by the first temperature sensor 165a in the dialysate outlet line 102) and/or a second temperature T₂ (here by the second temperature sensor 165b in the dialysis liquid inlet line 104). The temperature difference T₂-T₁ reflects the influence of the environment conditions, typically of the ambient temperature value T_{Umg}, on the temperature of the dialysis liquid or of the dialysate.

In the example of FIG. 2, calibration is performed without the dialyzer 303. In some embodiments, calibration may also be performed with the dialyzer 303, but can be performed without the patient, as the patient's blood temperature could distort the measurement result.

The calibration with short circuit line 350 described above is the fastest and, from a risk point of view, the least critical, since in this, only the radiation behavior of the tubes is included in the measurement.

The measurement would be most accurate if the dialyzer 303 were also connected, since its radiation pattern would then also be compensated for. This would mean additional work during the preparation phase, but is likewise covered by the disclosure.

FIG. 3 shows schematically simplified a control device or closed-loop control device 150 with temperature data streams.

A dialysis liquid flow along the heating device 162, the second temperature sensor 165b, through the dialyzer 303 or the resulting dialysate flow towards the first temperature sensor 165a is shown by a continuous arrow.

Determined temperatures are represented by dash-dot arrows in the example of FIG. 3.

The needed setpoint temperatures T_{D_soll}, T_{1_soll} for identifying a deviation of the temperature value T_{rech} from the setpoint temperature T_{D_soll} relevant or required at the dialyzer 303 and/or for identifying a deviation of the first temperature value T₁ from the setpoint temperature T_{1_soll} at the first temperature sensor 165a, are entered by the user or are stored in a storage device 151 provided for this purpose, and can be retrieved from there by the control device or closed-loop control device 150.

In addition to the components of the blood treatment apparatus 100, in the example of FIG. 3, the control device or closed-loop control device 150 is also in signal communication with an ambient temperature sensor 165c, which is optionally provided and suitable for determining the ambient temperature value Tung, to which the blood treatment apparatus 100 is exposed, and to transmit this value to the control device or closed-loop control device 150. Alternatively, this temperature can be retrieved from the ambient temperature sensor 165c by the control device or closed-loop control device 150.

Likewise, the two temperature sensors 165a and 165b, if both present or required for determining the temperature values T₁, T₂ (see previous figures), are directly or indirectly in signal communication with the control device or closed-loop control device 150.

In the example of FIG. 3, the control device or closed-loop control device 150 is configured in a design in order to calculate the calculated temperature value T_{rech} at the dialyzer 303. This may be based on the first temperature value T₁ and/or the second temperature value T₂ respectively of the first liquid, it may further be based on the temperature coefficient.

The calculation of the calculated temperature value T_{rech} at the blood treatment device or dialyzer 303 provides an estimated value of the actual temperature of the first liquid as it flows through the blood treatment device. Knowing said estimated value is used for determining a deviation between the estimated temperature at the blood treatment device and its setpoint value T_{D_soll} at this point. The deviation is used to prompt the increase or setting of the dialysis liquid temperature by the heating device 162 based on the deviation of the calculated temperature value T_{rech} from the setpoint value T_{D_soll}.

In this example, the influence of the ambient temperature value T_{Umg} should be homogeneous.

If a linear temperature drop is assumed for the respective line length L1, L2 of the line segments, a temperature coefficient Tₖ in the unit [°C/m] can be determined for these, e.g., as follows:

$${T}_{k} = \frac{( T 2 - T 1 )}{L 1 + L 2} ,$$

wherein the following applies:
T1 first temperature value measured by the first temperature sensor 165a downstream of the dialyzer 303
T2 second temperature value measured by the second temperature sensor 165b upstream of dialyzer 303
L1 length of the line segment, e.g., outside the blood treatment apparatus 100 upstream of the dialyzer 303
L2 length of the line segment, e.g., outside the blood treatment apparatus 100 downstream of the dialyzer 303

If a linear relationship between temperature drop and flow is assumed, the following applies for a reference flow of 500 ml/min:

$${T}_{k flow - compensated} = {T}_{k} * ( \frac{500 ml / min}{actual flow} )$$

Using the given formula, the control device or closed-loop control device 150 can calculate the temperature coefficient Tₖ and, via this or based thereon, by how many degrees the temperature to which the dialysis liquid has been brought so far by the heating device 162 is adjusted in order to compensate for the influence of the ambient temperature. This may lead to a reduction or an increase in the heating power.

In this, a calculated temperature value T_{rech} in the dialyzer 303 in an embodiment:

T_{rech}=T₂−L1*Tₖ

or

T_{rech}=T₁+L2*Tₖ

and for the temperature on the first temperature sensor 165a:

T₁=T₂−((L1+L2)*Tₖ)

If, for example, a setpoint temperature in the sense of a “desired temperature” of T_{D_soll}=37 °C is to prevail for the dialyzer 303, and if a temperature value T₂ of 37 °C is measured by the second temperature sensor 165b upstream of the blood treatment unit and a temperature loss of 1 °C is determined in each case over the lengths L1 and L2—assumed here to be of equal length—of the line sections, since the first temperature value T1 determined by the first temperature sensor 165a is 35 °C, then the calculated temperature value T_{rech} for the dialyzer 303 is 36 °C.

In order to compensate for the difference between T_{rech} and T_{D_soll}, the heating device 162 can be caused to heat the dialysis liquid more by just this difference so that the desired 37 °C is reached in the dialyzer 303. The control device or closed-loop control device 150 may be programmed to accordingly control or control in a closed-loop manner the heating device 162.

However, the example of FIG. 3 discloses yet another embodiment in which the control device or closed-loop control device 150 is configured to determine the first temperature value T₁ of the first liquid by the first temperature sensor 165a, in order to determine a deviation of the determined temperature value T₁ from the setpoint value T_{1_soll} for the setpoint temperature or desired temperature at this point, and to prompt the increase or the setting of the dialysis liquid temperature by the heating device 162 based on the deviation between the first temperature value T₁ and the setpoint value T_{1_soll}. This may be carried out by closed-loop control in that again the first temperature value T₁ is determined and compared to the setpoint value T_{1_soll} after a change of the heating power by the heating device 162, etc. In this procedure, consideration of an ambient temperature value T_{Umg} can be dispensed with, as can consideration of the second temperature value T₂.

As is seen from the aforementioned statements, device-internal influences on the temperature which effect, e.g., the dialysis liquid after it has flowed past the second temperature sensor 165b are neglected here. This is harmless to a first approximation. However, in embodiments other than those discussed above, such device-internal influences are also considered. This may be done, e.g., when determining the temperature coefficient Tₖ.

Values, setpoint values, coefficients and the like disclosed herein may optionally be determined or calculated by the control device or closed-loop control device 150 within the scope of the foregoing disclosure, for example by a corresponding computing device which is comprised by or connected to the control device or closed-loop control device 150. These values, setpoint values, coefficients and the like may alternatively be taken from a storage device 151 by the latter, respectively. There, these values, setpoint values, coefficients and the like may have been stored after they were first determined by the control device or closed-loop control device 150 or the computing device in a previous step or at an earlier point in time.

Although the present disclosure has been explained herein with emphasis on the example of a blood treatment apparatus which uses a dialyzer for blood treatment, the present disclosure is not limited thereto. Its use in other fields of medical technology, which does not serve for the treatment of renally impaired persons is also encompassed by the present disclosure.

### LIST OF REFERENCE NUMERALS

100 blood treatment apparatus
101 blood pump
102 dialysate outlet line
102a connector
104 dialysis liquid inlet line
104a connector
105 substitute fluid line
107 predilution valve
107a line associated with the predilution valve
109 postdilution valve
109a line associated with the postdilution valve
111 substitute fluid line
131 ultrafiltration pump
150 control device or closed-loop control device
151 storage device
153 drainage
155 water source
157 heat exchanger
159 first flow pump
161 device for balancing
162 heating device
163 mixing device
163a conductivity sensor
163b conductivity sensor
165a first temperature sensor
165b second temperature sensor
165c ambient temperature sensor
166 concentrate supply
167 leakage sensor
168 concentrate supply
169 second flow pump
171 pump, sodium pump
173 pump, bicarbonate pump
300 extracorporeal blood circuit
301 first line (arterial line section)
302 (first) tube clamp
303 blood filter or dialyzer
303a dialysis liquid chamber
303b blood chamber
303c semipermeable membrane
304a dialysate port
304b dialysate port
305 second line (venous line section)
306 (second) tube clamp
315 detector
317 single-needle chamber
318 deaeration device
319 detector
325 addition site for Heparin
329 venous blood chamber (optional)
350 short circuit line; short circuit
360 housing wall
F1 filter
F2 filter
A container; A-concentrate; sodium
B container; B-concentrate; bicarbonate
K compressed pressure source; compressor
L1 length of first line segment
L2 length of second line segment
P pressure measurement sites
PS1 arterial pressure sensor (optional)
PS2 arterial pressure sensor (optional)
PS3 pressure sensor (optional)
PS4 pressure sensor for measuring the filtrate pressure (optional)
PS5 pressure sensor for measuring the pressure in the dialysis liquid inlet line
T₁ first temperature value
T₂ second temperature value
T_{D} temperature in the dialyzer
Tₖ temperature coefficient
T_{rech} calculated temperature value
T_{D_soll} setpoint value for the temperature at the blood treatment device
T_{1_soll} setpoint value for the temperature at the first temperature sensor
T_{Umg} ambient temperature value
V valves
V24 valve
V25 valve
VB bypass valve
Y Y-connector

1-22. (canceled)

23. A control device or closed-loop control device configured to control or control in a closed-loop manner operation of a blood treatment apparatus for treating a patient during a blood treatment session, in which the blood treatment apparatus is connected to an extracorporeal blood circuit and a blood treatment device, wherein
the blood treatment apparatus comprises:
a dialysis liquid inlet line and a dialysate outlet line arranged to respectively supply or drain dialysis liquid to or from the blood treatment device during the blood treatment session,
a heating device for heating the dialysis liquid prior to, or during, the blood treatment session, and
at least a first temperature sensor arranged downstream of the blood treatment device and/or downstream of the dialysis liquid inlet line, wherein the at least first sensor is configured to determine a first temperature value of a first liquid being advanced along the dialysis liquid inlet line; and
the control device or closed-loop control device is configured to increase or set a temperature of the dialysis liquid using the heating device based on the first temperature value determined by the first temperature sensor.

24. The control device or closed-loop control device of claim 23, wherein the control device or closed-loop control device is configured to determine a deviation of the first temperature value of the first liquid from a setpoint value.

25. The control device or closed-loop control device of claim 23, wherein the control device or closed-loop control device is configured to determine the first temperature value only downstream of the blood treatment device.

26. The control device or closed-loop control device of claim 23, wherein the control device or closed-loop control device is configured to, during or prior to the blood treatment session, further take into account an ambient temperature value determined by an ambient temperature sensor when increasing or setting the temperature to which the dialysis liquid is brought using the heating device.

27. The control device or closed-loop control device of claim 23, wherein the control device or closed-loop control device is configured to increase or set the temperature using the heating device additionally based on a second temperature value determined for the first liquid at least one of upstream of the blood treatment device or upstream of the dialysate outlet line by a second temperature sensor.

28. The control device or closed-loop control device of claim 27, wherein the control device or closed-loop control device is configured to determine the first temperature value and/or the second temperature value prior to a start of the blood treatment session.

29. The control device or closed-loop control device of claim 27, wherein the control device or closed-loop control device is configured to determine at least one of the first temperature value or the second temperature value during the blood treatment session.

30. The control device or closed-loop control device of claim 27, wherein the control device or closed-loop control device is configured to determine the first temperature value or second temperature value while the dialysis liquid inlet line and the dialysate outlet line are in fluid communication with each other and excluding the blood treatment device.

31. The control device or closed-loop control device of claim 27, wherein the control device or closed-loop control device is configured to determine at least one of the first temperature value or the second temperature value while the dialysis liquid inlet line and the dialysate outlet line are arranged including the blood treatment device.

32. The control device or closed-loop control device of claim 31, wherein when determining at least one of the first temperature value or the second temperature value there are not two liquids, being different from each other, present in the blood treatment device.

33. The control device or closed-loop control device of claim 27, further configured to:
calculate a calculated temperature value based on both the first temperature value and a second temperature value of the first liquid;
determine a deviation of the calculated temperature value at the blood treatment device from a setpoint value; and
prompt the increase or the setting of the dialysis liquid temperature by the heating device based on the deviation of the calculated temperature value from the setpoint value.

34. The control device or closed-loop control device of claim 33, wherein the control device or closed-loop control device is configured to determine the calculated temperature value at the blood treatment device based on at least one of the first temperature value or the second temperature value as well as on a temperature coefficient, wherein the temperature coefficient is determined for the dialysis liquid inlet line and/or for the dialysate outlet line.

35. The control device or closed-loop control device of claim 34, configured to determine the temperature coefficient.

36. The control device or closed-loop control device of claim 35, configured to determine the temperature coefficient related to at least one flow rate of the first liquid through or into (i) the blood treatment device, or (ii) a short circuit of the dialysis liquid inlet line and dialysate outlet line excluding or bypassing the blood treatment device.

37. The control device or closed-loop control device of claim 33, configured to increase or decrease the temperature of the dialysis liquid, during or prior to the blood treatment session, by the difference between the first temperature value and a setpoint value for the first temperature, or by at least a difference between the calculated temperature value and the setpoint value for the first temperature.

38. A blood treatment apparatus prepared for use with an extracorporeal blood circuit and a blood treatment device for treating a patient's blood during a blood treatment session, the blood treatment apparatus comprising:
at least one first receptacle for releasably receiving at least one section of the extracorporeal blood circuit;
at least one second receptacle for releasably receiving at least one section of the blood treatment device;
a dialysis liquid inlet line and a dialysate outlet line arranged to respectively supply and drain dialysate liquid to and from the blood treatment apparatus during the blood treatment session, or a third receptacle therefor;
a first temperature sensor arranged downstream of the blood treatment device and/or downstream of a dialysis liquid inlet line, wherein the first temperature sensor is configured to determine a first temperature value of a first liquid flowing through or resting in the dialysis liquid inlet line;
a heating device for heating the dialysis liquid prior to or during the blood treatment session; and
a control device or closed-loop control device configured to control or control in a closed-loop manner operation of the blood treatment apparatus, wherein the control device or closed-loop control device is configured to:
increase or set a temperature of the dialysis liquid using the heating device based on the first temperature value determined by the first temperature sensor.

39. The blood treatment apparatus of claim 38, further comprising a second temperature sensor configured to determining a second temperature value of the first liquid upstream of the blood treatment device or upstream of the dialysate outlet line.

40. The blood treatment apparatus of claim 38, further comprising, or connected to, an ambient temperature sensor for determining an ambient temperature value.

41. The blood treatment apparatus of claim 38, wherein the blood treatment apparatus comprises a dialysis apparatus, a hemodialysis apparatus, a hemofiltration apparatus, a hemodiafiltration apparatus, an apparatus for the acute renal replacement therapy, an apparatus for chronic renal replacement therapy, or an apparatus for the continuous renal replacement therapy.

42. A non-transitory digital storage medium configured to interact with a programmable computer system such that a conventional blood treatment apparatus is reprogrammed into a blood treatment apparatus of claim 38.

43. A computer program product having a program code stored on a non-transitory machine-readable carrier, for interacting with a programmable computer system such that a conventional blood treatment apparatus is reprogrammed into a blood treatment apparatus of claim 38.

44. A non-transitory computer program with a program code configured such that a conventional blood treatment apparatus is reprogrammed into a blood treatment apparatus of claim 38 when the computer program is running on a computer.
